(19) United States
(12) Patent Application Publication
(10) Pub. No.: US 2024/0115435 A1
(43) Pub. Date: Apr. 11, 2024

(54) ABSORBENT BODY WITH COMPOSITE ISOLATION SHEET

(71) Applicant: The Procter & Gamble Company, Cincinnati, OH (US)

(72) Inventors: Yoichiro YAMAMOTO, Guangzhou (CN); Fengchun YE, Beijing (CN); Hiroshi FUJIKAWA, Akashi (JP); Jiale HUANG, Guangzhou (CN); Yi YUAN, Beijing (CN)

(21) Appl. No.: 18/474,560

(22) Filed: Sep. 26, 2023

(30) Foreign Application Priority Data
Sep. 28, 2022 (WO) PCT/CN2022/122097

(51) Int. Cl. A61F 13/537; A61F 13/15; A61F 13/49

(52) U.S. Cl. CPC A61F 13/53747; A61F 13/15203; A61F 13/49011; A61F 13/49014; A61F 13/49019; A61F 13/53704; A61F 2013/530613

(57) ABSTRACT

Disclosed is an absorbent body for an absorbent article comprising 1) a water permeable topsheet; 2) a water impermeable backsheet; 3) an absorbent core disposed between the topsheet and the backsheet; and 4) a composite isolation sheet bonded to the wearer facing side of the topsheet.

### CROSS REFERENCE TO RELATED APPLICATION

This application claims priority, under 35 U.S.C. § 119, to PCT Patent Application PCT/CN2022/122097, filed on Sep. 28, 2022, the entire disclosure of which is hereby incorporated by reference.

### FIELD

This invention relates to absorbent bodies useful for absorbent articles such as diapers having a composite isolation sheet for isolating body exudates from the wearer's skin.

### BACKGROUND

Absorbent articles for personal hygiene, such as disposable diapers, disposable pants, and adult incontinence undergarments, are designed to absorb and contain various body exudates, including urine, menses, low viscosity fecal matter, and solid fecal matter. Fecal material is often difficult to remove from the skin of the user, in particular from sensitive skin such as of young babies and such as the skin around the genitals. Moreover, it is known that when fecal material and urine meet on the skin, this may cause irritation and redness of the skin and sometimes even dermatitis of the skin.

One of the solutions to reduce the fecal material on the skin is to provide a means to isolate the fecal material immediately after discharge, away from the skin. For example, diapers having an elasticized sheet closest to the wearer with an opening, through which the feces can pass to a void space between the sheet closest to the wearer and the remainder of the absorbent body, have been developed. The fecal material is then stored underneath the sheet closest to the wearer, away from the skin.

Absorbent articles with means to isolate the fecal material have been known in the art, such as in EP 357298A and JP 3130365B. While such absorbent articles have been known, they have shortcomings that have prevented significant commercial success. For example, those known in the art may be difficult to produce in today's high speed manufacturing process, as requiring elasticity along the shaped opening, or in various directions deviating from the machine direction.

Based on the foregoing, there is a need for an absorbent article having a composite isolation sheet for isolating body exudates from the wearer's skin while maintaining the performance of containment and wear comfort. There is also a need for such an absorbent article which may be economically manufactured.

### SUMMARY

The present invention is directed to an absorbent body for an absorbent article having a transverse direction, a longitudinal direction, a front end, a back end, a front region, a back region, a center region, a garment facing direction, and a wearer facing direction, comprising:

1) a water permeable topsheet;

2) a water impermeable backsheet;

3) an absorbent core disposed between the topsheet and the backsheet;

4) a composite isolation sheet bonded to the wearer facing side of the topsheet, the composite isolation sheet comprising:
a) a front portion in the front region having a front longitudinal end point and a pair of front opening edges, wherein the pair of front opening edges are not elasticized;
b) a center portion in the center region having a pair of side edges which are elasticized in the longitudinal direction;
c) a back portion in the back region having a back longitudinal end point and a pair of back opening edges wherein the pair of back opening edges are not elasticized;
d) a longitudinal elastic element disposed on at least one of the front portion and the back portion, wherein the longitudinal elastic element extends substantially in the longitudinal direction; and
e) an opening defined by the pair of front opening edges, the pair of side edges, and the pair of back opening edges, wherein the opening, upon wear, accommodates the anal and urethal orifice of the wearer.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings and which like designations are used to designate substantially identical elements, and in which:

FIG. 1 is schematic plan view of an embodiment of an absorbent body and absorbent article of the present invention showing the wearer facing side, and with any elastic elements fully stretched.

FIG. 2A is a transverse schematic cross section view taken along line A-A of FIG. 1 with the thickness exaggerated, and elastic elements contracted.

FIG. 2B is a transverse schematic cross section view taken along line B-B of FIG. 1 with the thickness exaggerated, and elastic elements contracted.

FIGS. 3A-3F are schematic cross section views of embodiments of the composite isolating sheet of the present invention.

### DEFINITIONS

As used herein, the following terms shall have the meaning specified thereafter:

“Absorbent article” refers to articles of wear which may be in the form of taped type diapers, pant type diapers, incontinent briefs, feminine hygiene garments, and the like. The “absorbent article” may be so configured to absorb and contain various exudates such as urine, feces, and menses discharged from the body. The “absorbent article” may refer to a combined merchandise of an outer cover adaptable to be joined with a separable disposable absorbent insert for providing absorbent and containment function, such as those disclosed in PCT publication WO 2011/087503A.

“Longitudinal” refers to a direction running substantially perpendicular from a waist edge to an opposing waist edge of the article and generally parallel to the maximum linear dimension of the article.

“Transverse” refers to a direction perpendicular to the longitudinal direction.

“Proximal” and “distal” refer respectively to the position closer or farther relative to the longitudinal center of the article.

“Wearer-facing” and “garment-facing” refer respectively to the relative location of an element or a surface of an element or group of elements. “Wearer-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

“Disposed” refers to an element being located in a particular place or position.

“Joined” refers to configurations whereby an element is directly secured to another element by affixing the element directly to the other element and to configurations whereby an element is indirectly secured to another element by affixing the element to intermediate member(s) which in turn are affixed to the other element.

“Film” refers to a sheet-like material wherein the length and width of the material far exceed the thickness of the material. Typically, films have a thickness of about 0.5 mm or less.

“Nonwoven”, nonwoven layer” or “nonwoven web” are used interchangeably to mean an engineered fibrous assembly, primarily planar, which has been given a designed level of structural integrity by physical and/or chemical means, excluding weaving, knitting or papermaking (ISO 9092:2019 definition). The directionally or randomly orientated fibers, are bonded by friction, and/or cohesion and/or adhesion. The fibers may be of natural or synthetic origin and may be staple or continuous filaments or be formed in situ. Commercially available fibers have diameters ranging from less than about 0.001 mm to more than about 0.2 mm and they come in several different forms such as short fibers (known as staple, or chopped), continuous single fibers (filaments or monofilaments), untwisted bundles of continuous filaments (tow), and twisted bundles of continuous filaments (yam). Nonwoven webs can be formed by many processes such as meltblowing, spunbonding, solvent spinning, electrospinning, carding and airlaying. The basis weight of nonwoven webs is usually expressed in grams per square meter (g/m² or gsm).

“Water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable”.

“Hydrophilic” describes surfaces of substrates which are wettable by aqueous fluids (e.g., aqueous body fluids) deposited on these substrates. Hydrophilicity and wettability are typically defined in terms of contact angle and the strike-through time of the fluids, for example through a nonwoven fabric. This is discussed in detail in the American Chemical Society publication entitled “Contact Angle, Wettability and Adhesion”, edited by Robert F. Gould (Copyright 1964). A surface of a substrate is said to be wetted by a fluid (i.e., hydrophilic) when either the contact angle between the fluid and the surface is less than 90°, or when the fluid tends to spread spontaneously across the surface of the substrate, both conditions are normally co-existing. Conversely, a substrate is considered to be “hydrophobic” if the contact angle is greater than 90° and the fluid does not spread spontaneously across the surface of the fiber.

“Extendibility” and “extensible” mean that the width or length of the component in a relaxed state can be extended or increased.

“Elasticated” and “elasticized” mean that a component comprises at least a portion made of elastic material.

“Elongatable material”, “extensible material”, or “stretchable material” are used interchangeably and refer to a material that, upon application of a biasing force, can stretch to an elongated length of at least about 110% of its relaxed, original length (i.e. can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

“Dimension”, “Length”, “Width”, “Pitch”, “Diameter”, “Aspect Ratio”, “Angle”, and “Area” of the article are all measured in a state wherein the article is extended to the Full Stretch Circumference W1 according to the “Whole Article Force Measurement” herein, and utilizing a ruler or a loupe, unless specified otherwise.

“Artwork” refers to a visual presentation to the naked eye, which is provided by printing or otherwise, and having a color. Printing includes various methods and apparatus well known to those skilled in the art such as lithographic, screen printing, flexographic, and gravure ink jet printing techniques.

“Color” or “Colored” as referred to herein includes any primary color except color white, i.e., black, red, blue, violet, orange, yellow, green, and indigo as well as any declination thereof or mixture thereof. The color white is defined as those colors having a L* value of at least 94, an a* value equal to 0±2, and a b* value equal to 0±2 according to the CIE L* a* b* color system.

### DETAILED DESCRIPTION

### Absorbent Body and Absorbent Article

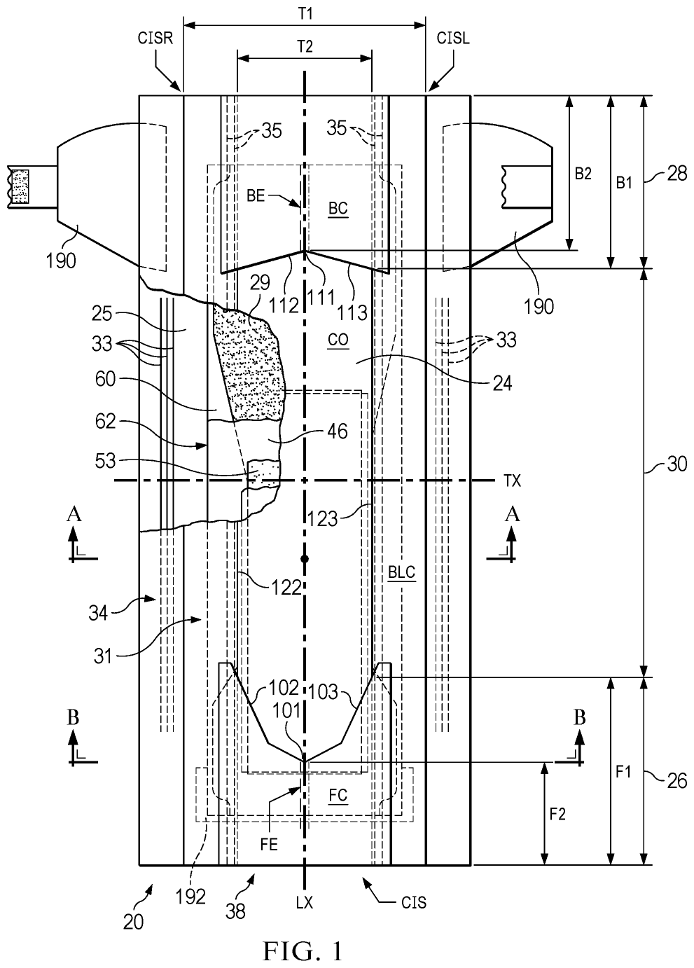

FIG. 1 is a schematic plan view of an absorbent article (20) of the taped type carrying the absorbent body (38) of the present invention. The absorbent article (20) and absorbent body (38) have a longitudinal centerline LX which also serves as the longitudinal axis, and a transverse centerline TX which also serves as the transverse axis, a front end and a back end. The absorbent body (38) has a wearer-facing surface, a garment facing surface, a liquid permeable topsheet, a liquid impermeable backsheet, an absorbent core disposed between the topsheet and the backsheet, and a composite isolation sheet bonded to the wearer facing side of the topsheet. The absorbent body (38) of the present invention may be assembled together with an application means, wherein the application means is selected from the group of a fastening means and an elastic belt. The exemplary absorbent article of FIG. 1 comprises a fastening means comprising a pair of elongate members (190) and a receiving member (192), the elongate members (190) transversely protruding from the left and right side edges of the back region and fastenable with the receiving member (192) disposed on the front region.

While not shown, the application means may also be an elastic belt, and the absorbent article (20) may take the form of a pant.

### Topsheet and Backsheet

The absorbent body (38) of the present invention comprises a water permeable topsheet (24) that may be positioned at least in partial contact or close proximity to a wearer. Suitable topsheets (24) may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. The topsheet (24) is generally supple, soft feeling, and non-irritating to a wearer's skin. The topsheet (24) is liquid permeable, permitting bodily fluids to readily penetrate through the thickness of the topsheet (24). One topsheet (24) useful herein is available from Fibertex NiLai, Malaysia with tradename H30501221 or FQN Hazlet NJ with tradename SB 1206169. Any portion of the topsheet (24) may be coated with a lotion or skin care composition as is known in the art. Examples of suitable lotions include those described in U.S. Pat. Nos. 5,607,760; 5,609,587; 5,635,191; and 5,643,588.

The absorbent body (38) of the present invention comprises a water impermeable backsheet (25) which is designed to prevent the exudates absorbed by and contained within the absorbent core (59) from soiling articles that may contact the absorbent article, such as bed sheets and undergarments. The backsheet (25) may be positioned such that it extends beyond the absorbent core (62) in both the longitudinal direction and the transverse direction. Suitable backsheet (25) materials include films such as those manufactured by Plaster Argentina with tradename PLBA NBBS 10-12GSM PR V1. Other suitable backsheet (25) materials may include breathable materials that permit vapors to escape from the absorbent article while still preventing exudates from passing through the backsheet (25). Exemplary breathable materials may include materials such as woven webs, nonwoven webs, composite materials such as film-coated nonwoven webs, and microporous films such as manufactured by Daika Japan with tradename MPF DKH-180 15G V7 and manufactured by Berry Nashville, TN with trademark BR-137P V13. Such breathable composite materials are described in greater detail in PCT Application No. WO 95/16746 and U.S. Pat. No. 5,865,823. Other breathable backsheets including nonwoven webs and apertured formed films are described in U.S. Pat. No. 5,571,096. An exemplary, suitable backsheet is disclosed in U.S. Pat. No. 6,107,537. Other suitable materials and/or manufacturing techniques may be used to provide a suitable backsheet including, but not limited to, surface treatments, particular film selections and processing, particular filament selections and processing, etc.

### Absorbent Core

Referring to FIG. 1, the absorbent body (38) of the present invention comprises an absorbent core (62) for absorbing and containing body exudates disposed on the wearer facing side. The absorbent core (62) may include an absorbent layer and an acquisition system (51). The absorbent layer is the region wherein absorbent materials (29) having a high retention capacity, such as superabsorbent polymers, are present. The absorbent layer may be substantially cellulose free. Alternatively, the absorbent layer may contain cellulose. There may be an absorbent layer mainly comprising cellulose, and another absorbent layer mainly comprising superabsorbent polymers.

Superabsorbent polymers of the absorbent layer may be disposed between first and second layers of material immobilized by a fibrous layer of thermoplastic adhesive material. The first and second layers of materials may be nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process.

The absorbent body (38) may also contain an acquisition system (51) for facilitating the acquisition and the distribution of body exudates, and may be placed between the topsheet (24) and the absorbent layer. The acquisition system (51) may include cellulosic fibers. The absorbent layers may be disposed in plurality in the absorbent core (62). Some portions of the absorbent layers may be configured to have substantially no absorbent material to form a channel or a plurality of channels. Channels may be useful for allowing the absorbent core (62) to bend upon swelling with fluids, such that the crotch region conforms to the wearer's body after swelling and prevent sagging of the article. The channels may also be formed in the acquisition system (51), and may be configured to at least partly match the channels of the absorbent layer in the thickness direction. The acquisition system (51) may comprise a liquid management layer (53) directly under the topsheet (24). The function of such a layer is to rapidly acquire the fluid from the topsheet (24) away from the wearer-facing side and/or to distribute over a larger area so it is more efficiently absorbed by the absorbent core. It is also possible that such a liquid management layer (53) may be placed between the backsheet (25) and the absorbent core. The liquid management layer may be a spunlace nonwoven comprising viscose, PET, CoPET/PET fibers, and combinations thereof.

The absorbent core (62) may comprise a high loft material encompassing superabsorbent polymers. The term “high loft” refers to low density bulky fabrics, as compared to flat, paper-like fabrics. High loft webs are characterized by a relatively high porosity. This means that there is a relatively high amount of void space in which superabsorbent polymer particles can be distributed. The high loft material (without the superabsorbent particles) of the invention may have a density at a pressure of 4.14 kPa (0.6 psi) below 0.20 g/cm³, in particular ranging from 0.05 g/cm³ to 0.15 g/cm³. The high loft layer (without the superabsorbent particles) may have a density at a pressure of 2.07 kPa (0.3 psi) below 0.20 g/cm³, in particular ranging from 0.02 g/cm³ to 0.15 g/cm³. The high loft layer (without the superabsorbent particles) of the invention may have a density at a pressure of 0.83 kPa (0.12 psi) below 0.15 g/cm³, in particular ranging from 0.01 g/cm³ to 0.15 g/cm³, and a basis weight of from 15 to 500 gsm, preferably 30-200 gsm, such as those described in US 2021/0361497 A1. The absorbent core (62) comprising high loft material encompassing superabsorbent polymers may also contain channels.

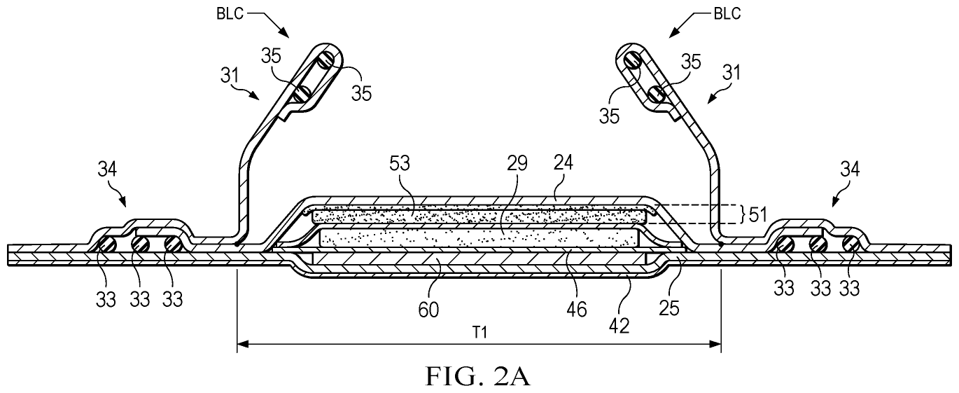

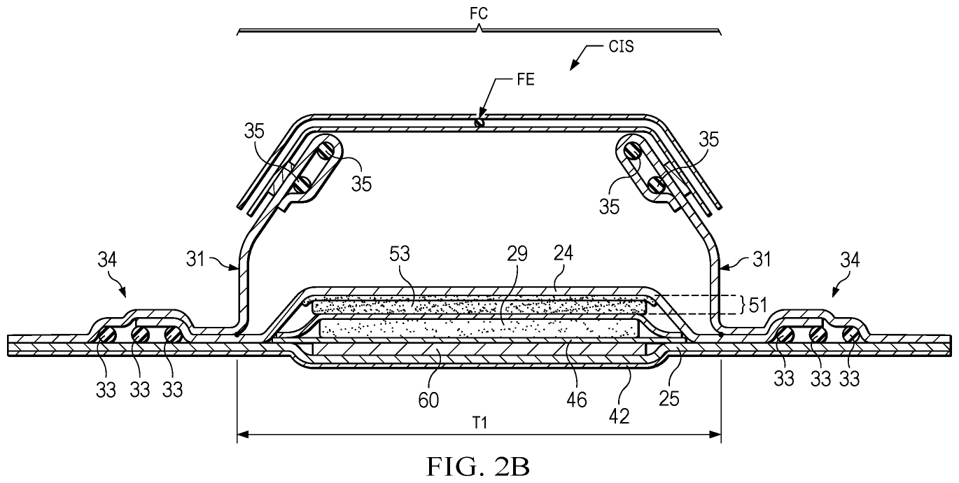

Alternatively, the absorbent core (62) may comprise an absorbent layer having superabsorbent polymers disposed between first and second layers of nonwoven material immobilized by a fibrous layer of thermoplastic adhesive material (not shown). The first and second layers of nonwoven materials may be relatively low basis weight nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process. Such an embodiment is exemplarily shown in New FIG. 2A. In such embodiments, a) the intermediate layer (60) may be hydrophobic and the lower substrate layer (46) may be hydrophilic; or b) the intermediate layer (60) and the lower substrate layer (46) may both be hydrophilic and the intermediate layer (60) may be less hydrophilic than the lower substrate layer (46); or c) the intermediate layer (60) and the lower substrate layer (46) may both be hydrophobic and the lower substrate layer (46) may be less hydrophobic than the intermediate layer (60).

### Composite Isolation Sheet

Referring to FIG. 1, the absorbent body (38) of the present invention comprises a composite isolation sheet (CIS) bonded to the wearer facing side of the topsheet for isolating body exudates from the wearer's skin. The composite isolation sheet (CIS) comprises: a) a front portion (FC) in the front region (26), b) a center portion (BLC) in the center region (30) having a pair of side edges which are elasticized in the longitudinal direction, c) a back portion (BC) in the back region (28), d) a longitudinal elastic element, and e) an opening (CO), wherein the opening (CO), upon wear, accommodates the anal and urethal orifice of the wearer. The region and area of the composite isolation sheet (CIS) is defined by the front end of the absorbent body, the back end of the absorbent body, a left side end (CISL), and a right side end (CISR), the left and right side ends (CISL, CISR) being bonded to the topsheet (24). Although the substrate for providing the composite isolation sheet (CIS) may extend into other elements of the absorbent body (38) such as the outer cuffs as described later, such other elements are not considered the composite isolation sheet (CIS). The center region (30) is defined by the line extending parallel to the transverse axis matching the front end of the pair of side edges (F1) and the line extending parallel to the transverse axis matching the back end of the pair of side edges (B1). The front region (26) extends from the line defining the center portion (BLC) on the front side (F1) to the front end of the absorbent body. The back region (28) extends from the line defining the center portion (BLC) on the back side (B1) to the back end of the absorbent body.

Still referring to FIG. 1, the front portion (FC) of the composite isolation sheet (CIS) has a front longitudinal end point (101) and a pair of front opening edges (102, 103), wherein the front opening edges (102, 103) are so configured such that the front longitudinal end point (101) is positioned at the front end of the opening (CO). The front opening edges (102, 103) may be curved, straight, or formed by a series of straight lines. The front portion (FC) has a longitudinal dimension (F1) which may be from about 22% to about 50% of the longitudinal dimension (L1) of the absorbent body (38). The distance from the front longitudinal end point (101) to the front end, when fully stretched in case a longitudinal elastic body is present, is represented by F2. The dimensions F1, F2, and the shape of the front opening edges (102, 103) may be so configured such that, when the front longitudinal elastic element is fully stretched, the opening (CO) area in the front region (26) is from about 5% to about 30% of the composite isolation sheet (CIS) in the front region (26). Similarly, the back portion (BC) of the composite isolation sheet (CIS) has a back longitudinal end point (111) and a pair of back opening edges (112, 113), wherein the back opening edges (112, 113) are so configured such that the back longitudinal end point (111) is positioned at the back end of the opening (CO). The back opening edges (112, 113) may be curved, straight, or formed by a series of straight lines. The back portion (BC) has a longitudinal dimension (B1) which may be from about 5% to about 35% of the longitudinal dimension of the absorbent body (L1). The distance from the back longitudinal end point (111) to the back end, when fully stretched in case a longitudinal elastic body is present, is represented by B2. The dimensions B1, B2, and the shape of the back opening edges (112, 113) may be so configured such that the opening (CO) area in the back region (28) is smaller than the opening area in the front region (26). In the back portion (BC), B1 may equal B2.

Still referring to FIG. 1, the center portion (BLC) in the center region (30) is positioned between the front portion (FC) and the back portion (BC) and forms a pair of side edges which are elasticized in the longitudinal direction along the longitudinally extending side edges (122, 123) of the opening (CO) of the composite isolation sheet (CIS). The pair of side edges (122, 123) of the center portion (BLC) are elasticized by longitudinally extending elastic bodies (35) disposed adjacent the side edges (122, 123). The elastic bodies for elasticizing the side edges (122, 123) of the center portion (BLC) may be provided in a plurality of elastic strands (35). The composite isolation sheet (CIS) in the center portion (BLC) may have a transverse dimension (T1) between the left and right side ends (CISL, CISR) of from about 120 mm to about 170 mm. Such transverse dimension may be the same for the entire composite isolation sheet (CIS). The transverse distance (T2) between the side edges (122, 123), when the center portions (BLC) are completely laid flat on the topsheet, may be from about 60 mm to about 140 mm.

Still referring to FIG. 1, the composite isolation sheet (CIS) further comprises a longitudinal elastic element (FE, BE) disposed on at least one of the front portion (FC) and the back portion (BC) wherein the longitudinal elastic element extends substantially in the longitudinal direction. The longitudinal elastic element may be disposed only on the front portion (FC). The longitudinal elastic element may be disposed only on the back portion (BC).

The longitudinal elastic element may be disposed on both the front portion (FC) and the back portion (BC). The longitudinal elastic element may be provided in a single, or a plurality of longitudinal elastic body/bodies. The longitudinal elastic body may be an elastic ribbon or an elastic strand. When the longitudinal elastic element is a single elastic ribbon or strand, the longitudinal elastic body (FE, BE) may be disposed on and extending along the longitudinal axis (LX) between the front longitudinal end point (101) towards the front end, or the back longitudinal end point (111) towards the back end. In FIG. 1, the front longitudinal elastic body (FE) and back longitudinal elastic body (BE) are each depicted as matching the longitudinal axis (LX). While not shown, the longitudinal elastic element may be in a pair and disposed in a symmetric position against the longitudinal axis (LX) on the left and right portions of the front portion (FC). While not shown, the longitudinal elastic element may be in a pair and disposed in a symmetric position against the longitudinal axis (LX) on the left and right portions of the back portion (BC). While not shown, the longitudinal elastic element may be in 3 bodies and disposed along the longitudinal axis (LX) as well as in a symmetric position against the longitudinal axis (LX) on the left and right portions of the front portion (FC). In a similar manner, 3 longitudinal elastic bodies may be provided on the back portion (BC). Regardless of the number and position, any longitudinal elastic element disposed on the front portion (FC) or the back portion (BC) extends substantially in the longitudinal direction.

Referring to FIG. 1, when disposed on the front portion (FC), the longitudinal elastic body (FE) may extend from the front longitudinal end point (101) towards the front end. The front longitudinal elastic body (FE) may extend for only a portion of F2. While not shown, the longitudinal elastic body (FE) may extend for the entirety of F2, or in positions closer to the front end, or in the middle of the line extending between the front longitudinal end point (101) and the front end. The distance from the front longitudinal end point (101) to the front end, when fully contracted by the front longitudinal elastic body (FE), is represented by F3. The force provided by the front longitudinal elastic body (FE) may be such so that F3 may be from about 30% to about 85% of F2. When disposed on the back portion (BC), the longitudinal elastic body (BE) may extend from the back longitudinal end point (111) towards the back end. The back longitudinal elastic body (BE) may extend for only a portion of B2. While not shown, the longitudinal elastic body (BE) may extend for the entirety of B2, or in positions closer to the back end, or in the middle of the line extending between the back longitudinal end point (111) and the back end. The distance from the back longitudinal end point (111) to the back end, when fully contracted by the back longitudinal elastic body (BE), is represented by B3. The force provided by the back longitudinal elastic body (BE) may be such so that B3 may be from about 30% to about 85% of B2.

Accordingly, still referring to FIG. 1, the opening (CO) of the composite isolation sheet (CIS) is defined by the pair of front opening edges (102, 103), the pair of side edges (122, 123), and the pair of back opening edges (112, 113). In that the composite isolation sheet (CIS) holds tension provided by the combination of elastic bodies (35) of the side edges and the longitudinal elastic element (FE, BE) when contracted upon wear, the composite isolation sheet (CIS) is raised from the topsheet (24). The dimension of the opening and the tension provided by the elastic bodies (35) of the side edges and the longitudinal elastic element are adjusted so that, upon wear, the opening (CO) accommodates the anal and urethal orifice of the wearer. Referring to FIG. 2A representing the schematic cross section of FIG. 1 taken along line A-A in the center portion (BLC), only the elasticized side edges (122, 123) are observed. Referring to FIG. 2B representing the schematic cross section of FIG. 1 taken along line B-B in the front portion (FC), the front portion and optional front longitudinal elastic body (FE) are also observed. The composite isolation sheet (CIS) of the present invention is devoid of elasticity along the pair of front opening edges (102, 103) as well as the pair of back opening edges (112, 113). Even though there is such absence of elasticity around certain portions of the periphery of the opening (CO), the composite isolation sheet (CIS) may be provided sufficient tension by the combination of the elasticized side edges (122, 123) and the longitudinal elastic element. Such configuration of the composite isolation sheet (CIS) enables providing elasticization generally parallel to the longitudinal axis (LX) of the absorbent body. Such configuration of the composite isolation sheet (CIS) of the present invention is advantageous in that, when the machine direction for manufacturing the absorbent body matches the longitudinal direction, any elastic body to be disposed on the composite isolation sheet (CIS) may also be advanced in the machine direction for attachment.

Material for making the composite isolation sheet (CIS) may be made from a substantially liquid impervious material. The material may be an SMS nonwoven or an SMMS nonwoven material, or a nonwoven component layer comprising fine fibers having an average diameter of less than 1 micron. One useful combination of nonwoven fabric webs may include spunbond, meltblown, spunbond (“SMS”) webs comprising outer layers of spunbond thermoplastics (e.g., polyolefins) and an interior layer of meltblown thermoplastics. Suitable composite isolation sheet material useful herein include those of SMS type available from Toray Polytech Nantong China with tradename LIVSEN SMS 13, available from FQN Hazlet NJ with tradename SM15009270, and available from Fibertex Aalborg Denmark with tradename B10160HS. The material may be treated, by region or in part of a region, with a lotion or a hydrophobic surface coating to provide various physical properties. Material for making the composite isolation sheet (CIS) may have a hydrostatic head of greater than about 2 mbar, or greater than about 3 mbar, or greater than about 4 mbar. The material may have a hydrostatic head of less than about 200 mbar, or less than about 100 mbar, or less than about 75 mbar, or less than about 50 mbar, or less than about 25 mbar, or less than about 15 mbar. The material may have an opacity of from about 15% to about 50% hunter opacity, or from about 20% to about 45% hunter opacity. The material may have an opacity of from about 45% to about 75% hunter opacity; or from about 50% to about 70% hunter opacity. The material may have an air permeability of less than about 50 m³/m²/min; or less than about 45 m³/m²/min. The material may have an air permeability of greater than about 5 m³/m²/min; or greater than about 10 m³/m²/min; or greater than about 15 m³/m²/min; or greater than about 20 m³/m²/min.

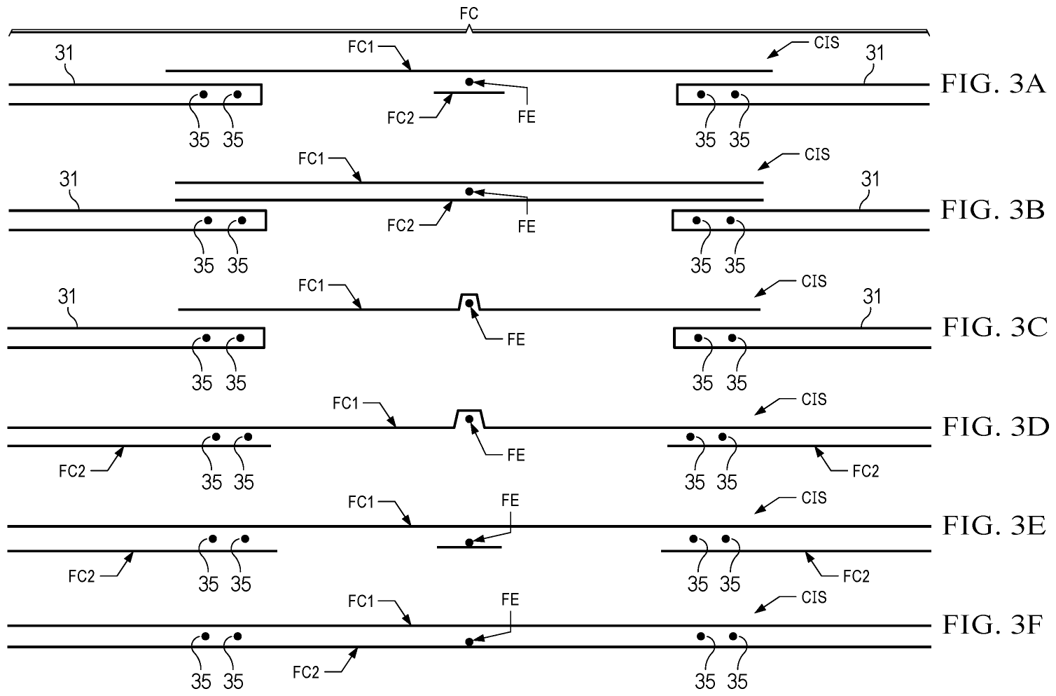

Referring to FIGS. 3A-3F, various configurations for forming the composite isolation sheet (CIS) are disclosed. FIGS. 3A-3F are schematic cross section views taken at line B-B of FIG. 1, with the substrates for making the composite isolating sheet (CIS) extended laterally and detached from the remainder of the absorbent body (38).

FIGS. 3A-3C are related to configurations wherein the center portion (BLC) is formed by a different part from the front and/or back portions (26, 28). In these configurations, the center portion (BLC) may have a configuration similar to an inner cuff extending in the longitudinal direction, wherein the inner cuff part (31) may extend the entire length of the absorbent body (38). When taking such configuration for the center portion (BLC), at least the region matching the center portion (BLC) is in active elasticity. When taking such configuration for the center portion (BLC), the inner cuff part (31) overlapping the front and back portions (FC, BC) may be devoid of active elasticity, particularly along the front and back ends of the absorbent core (38).

Referring to FIGS. 3A-3C, the front portion (FC) is formed by a part that is bonded on the wearer-facing side of the inner cuff part (31). The back portion (BC) may be formed in a similar manner. The substrate for forming the front and back portions (FC, BC) may be a continuous part which is connected in the longitudinal direction along the inner cuff part (31), or may be separate front and back parts that are not connected. Referring to FIG. 1, the substrate for forming the front and back portions (FC, BC) may be separate front and back parts that are not connected. By providing the front and back portions (FC, BC) formed by a continuous part, the center portion (BLC) may be provided in multiple substrate layers, and provide improved protection. By providing the front and back portions (FC, BC) formed in separate front and back parts, material may be saved, and the center portion (BLC) may have improved breathability.

Referring to FIG. 3A, when a front longitudinal elastic body (FE) is comprised in the front portion (FC), the front longitudinal elastic body (FE) may be disposed between an outer front portion sheet (FC1) and an inner front portion sheet (FC2). The inner front portion sheet (FC2) may have a shorter lateral dimension than the outer front portion sheet (FC1) as in FIG. 3A. Referring to FIG. 3C, the front longitudinal elastic body (FE) may be wrapped in a single layer of material. Such configurations of FIG. 3A or 3C may provide the front portion (FC) thin and breathable. Referring to FIGS. 2B and 3B, the inner front portion sheet (FC2) may have the same lateral dimension as the outer front portion sheet (FC1). Such a configuration enables secure attachment of the front longitudinal elastic body (FE), and further provides improved protection in this area.

FIGS. 3D-3F are related to configurations wherein the center portion (BLC) and at least the front portion (FC) are formed by the same integral part. In these configurations, the elasticizing of the side edges of the center portion (BLC) and the front longitudinal elastic body (FE) may be provided between the outer front portion sheet (FC1) and the inner front portion sheet (FC2).

For any of the configurations of FIGS. 3A-3F, the back portion (BC) may be formed in the same way as the front portion (FC) as described above.

### Other Components

Referring to FIGS. 1 and 2A, the absorbent body (38) of the present invention may further comprise components that improve leakage prevention, wearability, fit, or aesthetic aspects of the resulting absorbent article.

The absorbent body (38) may comprise gasketing leg cuffs (34). The gasketing leg cuffs (34) may be at least partially enclosed between the topsheet (24) and the backsheet (25), and may be placed transversely outward relative to the composite isolation sheet (CIS). The gasketing leg cuffs (34) may provide sealing around the thighs of the wearer. Usually each gasketing leg cuff (34) will comprise one or more elastic string or elastic element (33) comprised in the absorbent body (38) for example between the topsheet (24) and backsheet (25) in the area of the leg openings. The substrate for making the composite isolation sheet (CIS) may extend transversely and be utilized for forming the gasketing leg cuffs (34).

The absorbent body (38) of the present invention may comprise an outer cover (42) located on the garment-facing side of the backsheet (25). The outer cover (42) may be made of a soft, non-woven material. The outer cover (42) and the backsheet (25) may be joined together by adhesive or any other suitable material or method. A particularly suitable outer cover (42) is available from Fibertex NiLai Malaysia with tradename A10160EJ—MALAYSIA and available from FQN Hazlet NJ with tradename SM1104174.

The absorbent core (62) may comprise an intermediate layer (60) between the layer of absorbent material and the backsheet (25). The intermediate layer (60) may be in direct contact with the layer of absorbent material (29) and with the backsheet (25). The intermediate layer (60) may be useful as a masking layer to isolate the superabsorbent polymer particles in the layer of absorbent material from the backsheet (25), thus reducing graininess feeling and improving the tactile properties of the garment-facing side of the article, especially for absorbent core (62) containing a high level of superabsorbent polymer particles. The intermediate layer (60) may also isolate the exudates which have been absorbed in the layer of absorbent material from the garment-facing side of the article, as this may be visually unpleasant to the caregiver. Thus by having an intermediate layer with a relatively high opacity, stains in the layer of absorbent material (e.g. from urine or feces) can be concealed from view, when looking at the backsheet (25) of the absorbent article during use. The hunter opacity in the dry state of the intermediate layer may be at least 25%, or at least 40%, or at least 50%, or at least 70%. The intermediate layer (60) can also help reduce the residual moisture in contact with the backsheet (25), which may lead to cold/wet feeling for the caregiver, or may lead to the wearer mistaking the cold/wet feeling as liquid leaking out of the absorbent article. The intermediate layer (60) may also serve as a temporary reservoir for liquid that had not been absorbed fast enough by the layer of absorbent material.

### Opacity Method

Opacity is measured using a 0° illumination/45° detection, circumferential optical geometry, spectrophotometer with a computer interface such as the HunterLab LabScan XE running Universal Software (available from Hunter Associates Laboratory Inc., Reston, VA) or equivalent instrument. Instrument calibration and measurements are made using the standard white and black calibration plates provided by the vendor. All testing is performed in a room maintained at 23±2° C. and 50±2% relative humidity.

The spectrophotometer is configured for the XYZ color scale, D65 illuminant, 10° standard observers, with UV filter set to nominal. The instrument is standardized according to the manufacturer's procedures using the 0.7 inch port size and 0.5 inch area view. After calibration, the software is set to the Y opacity procedure which prompts the operator to cover the sample with either the white or black calibration tile during the measurement.

Articles are pre-conditioned at 23° C.±2 C.° and 50%±2% relative humidity for two hours prior to testing. To obtain a specimen, the article is stretched flat on a bench, body facing surface upward, and the total longitudinal length of the article is measured. A testing site is selected at the longitudinal midpoint of the article or the part. Using scissors, a test specimen is cut in 60 mm square or as close as possible to 60 mm. Any elastic members are removed.

The specimen is placed over the measurement port. The specimen should completely cover the port with the surface corresponding to the wearer-facing surface of the specimen directed toward the port. The specimen is gently extended until taut in its longitudinal direction so that the specimen lies flat against the port plate. Adhesive tape is applied to secure the specimen to the port plate in its extended state for testing. Tape should not cover any portion of the measurement port. The specimen is then covered with the white standard plate. A reading is taken, then the white tile is removed and replaced with the black standard tile without moving the specimen. A second reading is taken, and the opacity is calculated as follows:

Opacity=(Yvalue(black backing)/Yvalue(white backing))×100

Specimens from five identical articles are analyzed and their opacity results recorded. The average opacity is calculated and reported to the nearest 0.01%.

### Air Permeability Test

Air permeability is tested using a TexTest FX3300 Air Permeability Tester (available from Advanced Testing Instruments, Greer, SC) with a custom made 1 cm² circular aperture (also available from Advanced Testing Instruments) or equivalent instrument. The instrument is calibrated according to the manufacturer's procedures. All testing is performed in a room maintained at 23° C.±2 C.° and 50%±2% relative humidity.

The articles are pre-conditioned at 23° C.±2 C.° and 50%±2% relative humidity for two hours prior to testing. To obtain a specimen, the article is stretched flat on a bench, body facing surface upward, and the total longitudinal length of the article is measured. A testing site is selected at the longitudinal midpoint of the article or the part. Using scissors, a test specimen is cut in 30 mm square or as close as possible to 30 mm. Any elastic members are removed.

The specimen is centered over the measurement port. The specimen should completely cover the port with the surface corresponding to the wearer-facing surface of the specimen directed toward the port. The specimen is gently extended in its longitudinal direction until taut so that the specimen lies flat across the port. Adhesive tape is applied to secure the specimen across the port in its extended state for testing. Tape should not cover any portion of the measurement port. The test pressure is set to allow air to pass through the specimen. For non-woven specimen the pressure is set for 125 Pa and for specimen containing films 2125 Pa is used. The sample ring is closed and the measuring range is adjusted until the range indicator shows green to indicate that the measurement is within the accepted limits of the instrument. The air permeability is recorded to the nearest 0.1 m3/m2/min.

### Hydrostatic Head Test

Hydrostatic head is tested using a TexTest FX3000 Hydrostatic Head Tester (available from Advanced Testing Instruments, Greer, SC) with a custom made 1.5 cm2 circular measurement port (also available from Advanced Testing Instruments). Two annular sleeve rings, the same dimensions as the gaskets around the measurement ports, are cut from the standard protective sleeves for fine nonwovens (part FX3000-NWH, available from Advanced Testing Instruments). The sleeve rings are then adhered with two-sided adhesive tape to the sample facing surfaces of the upper and lower gaskets of the TexTest instrument to protect the specimen during clamping. Standardize the instrument according to the manufacturer's procedures. All testing is performed in a room maintained at about 23° C.±2 C.° and about 50%±2% relative humidity.

Precondition the articles at about 23° C.±2 C.° and about 50%±2% relative humidity for two hours prior to testing. To obtain a specimen, lay the article stretched flat on a bench, body facing surface upward, and measure the total longitudinal length of the article. A testing site is selected at the longitudinal midpoint of the article or the part. Using scissors, a test specimen is cut in 70 mm square or as close as possible to 70 mm. Any elastic members are removed.

Place the specimen centered over the port of the upper test head. The specimen should completely cover the port with the surface corresponding to the garment-facing surface of the specimen directed toward the port (wearer-facing surface will then be facing the water). Gently extend the specimen taut in its longitudinal direction so that the specimen lies flat against the upper test plate. Adhesive tape is applied to secure the specimen to the test plate in its extended state for testing. Tape should not cover any portion of the measurement port.

Fill the TexTest syringe with distilled water, adding the water through the measurement port of the lower test plate. The water level should be filled to the top of the lower gasket. Mount the upper test head onto the instrument and lower the test head to make a seal around the specimen. The test speed is set to 3 mbar/min for samples that have a hydrostatic head of 50 mbar or less and a speed of 60 mbar/min for samples with a hydrostatic head above 50 mbar. Start the test and observe the specimen surface to detect water droplets penetrating the surface. The test is terminated when one drop is detected on the surface of the specimen or the pressure exceeds 200 mbar. Record the pressure to the nearest 0.5 mbar or record as >200 mbar if there was no penetration detected.

A total of five identical articles are analyzed and their hydrostatic head results recorded. Calculate and report the average hydrostatic head report to the nearest 0.1 mbar.

### EXAMPLE

Absorbent article Examples 1-2 and Comparative Example 1 were obtained as such.

### Comparative Example 1

“Pampers [symbol] (Kangaroo Taped)” Size 4 purchased in 2022 in PRC.

### Example 1

Taped type absorbent article of Size 4 having an overall configuration similar to Comparative Example 1, and also having a front portion and a back portion as in FIGS. 1, 2A, and 2B, with dimensions as in Table 1 below.

### Example 2

Taped type absorbent article of Size 4 having an overall configuration similar to Comparative Example 1, and also having a front portion and back a portion as in FIGS. 1, 2A, and 2B, however with a back portion of different configuration and devoid of back longitudinal elastic body, with dimensions as in Table 1 below.

TABLE 1

| Dimension | Example 1 | Example 2 |
|---|---|---|
| Longitudinal dimension of the | 480 | 480 |
| absorbent body L1 (mm) | | |
| Longitudinal dimension of | 170 | 170 |
| the front portion F1 (mm) | | |
| F1/L1 (%) | 35.4 | 35.4 |
| Distance from front longitudinal | 100 | 100 |
| end point to front end when front | | |
| longitudinal elastic body | | |
| fully stretched F2 (mm) | | |
| Distance from front longitudinal | 70 | 70 |
| end point to front end when front | | |
| longitudinal elastic body fully | | |
| contracted F3 (mm) | | |
| F2/F3 (%) | 70 | 70 |
| Area of opening of composite | 13.2 | 13.2 |
| isolation sheet in the front region (%) | | |
| Transverse dimension of the | 142 | 142 |
| composite isolation sheet T1 (mm) | | |
| Distance between the side edges | 74 | 74 |
| of the center portion T2 (mm) | | |
| Longitudinal dimension | 155 | 80 |
| of the back portion B1 (mm) | | |
| B1/L1 (%) | 32.3 | 16.7 |
| Distance from back longitudinal | 135 | 75 |
| end point to back end when back | | |
| longitudinal elastic body | | |
| fully stretched B2 (mm) | | |
| Distance from back longitudinal | 102 | NA |
| end point to back end when back | | |
| longitudinal elastic body | | |
| fully contracted B3 (mm) | | |
| B2/B3 (%) | 75.6 | NA |

The article of Examples 1 and 2, when worn, provide the composite isolation sheet raised from the topsheet, with the opening accommodating the anal and urethal orifice of the intended wearer. Examples 1 and 2 provide good isolation of body exudates from the wearer's skin, while maintaining the performance of containment and wear comfort of Comparative Example 1. The articles of Example 1 and 2 may be manufactured in approximately the same speed as manufacturing Comparative Example 1.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Further, every numerical range given throughout this specification includes every narrower numerical range that falls within such broader numerical range.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent body for an absorbent article having a transverse direction, a longitudinal direction, a front end, a back end, a front region, a back region, a center region, a garment facing side, and a wearer facing side, comprising:
1) a water permeable topsheet;
2) a water impermeable backsheet;
3) an absorbent core disposed between the topsheet and the backsheet;
4) a composite isolation sheet bonded to the wearer facing side of the topsheet, the composite isolation sheet comprising:
a) a front portion in the front region having a front longitudinal end point and a pair of front opening edges, wherein the pair of front opening edges are not elasticized;
b) a center portion in the center region having a pair of side edges which are elasticized in the longitudinal direction;
c) a back portion in the back region having a back longitudinal end point and a pair of back opening edges, wherein the pair of back opening edges are not elasticized;
d) a longitudinal elastic element disposed on at least one of the front portion and the back portion, wherein the longitudinal elastic element extends substantially in the longitudinal direction; and
e) an opening defined by the pair of front opening edges, the pair of side edges, and the pair of back opening edges, wherein the opening, upon wear, accommodates the anal and urethal orifice of a wearer.

2. The absorbent body of claim 1, wherein the front portion has a longitudinal dimension F1 of from about 22% to about 50% of the longitudinal dimension of the absorbent body.

3. The absorbent body of claim 2, wherein the longitudinal elastic element is disposed on the front portion.

4. The absorbent body of claim 3, wherein, when the front longitudinal elastic element is fully stretched, the opening area in the front region is from about 5% to about 30% of the composite isolation sheet in the front region.

5. The absorbent body of claim 3, wherein, the distance from the front longitudinal end point to the front end when the front longitudinal elastic element is fully stretched is represented by F2, and the distance from the front longitudinal end point to the front end when the front longitudinal elastic body is fully contracted is represented by F3, F3 is from about 30% to about 85% of F2.

6. The absorbent body of claim 3, wherein the front longitudinal elastic element is an elastic body extending along a longitudinal axis between the front longitudinal end point towards the front end.

7. The absorbent body of claim 1, wherein the back portion has a longitudinal dimension B1 of from about 5% to about 35% of the longitudinal dimension of the absorbent body.

8. The absorbent body of claim 7, wherein the longitudinal elastic element is disposed on the back portion.

9. The absorbent body of claim 8, wherein, the distance from the back longitudinal end point to the back end when the back longitudinal elastic element is fully stretched is represented by B2, and the distance from the back longitudinal end point to the back end when the back longitudinal elastic body is fully contracted is represented by B3, B3 is from about 30% to about 85% of B2.

10. The absorbent body of claim 8, wherein the back longitudinal elastic element is an elastic body extending along a longitudinal axis between the back longitudinal end point towards the back end.

11. The absorbent body of claim 7, wherein the back portion is devoid of a longitudinal elastic element.

12. The absorbent body of claim 1, wherein the composite isolation sheet comprises an inner cuff part and a continuous part, wherein the pair of elasticized side edges are formed by the inner cuff portion, wherein the continuous part forms the front portion and the back portion, and wherein the continuous part is connected in the longitudinal direction along the inner cuff part.

13. The absorbent body of claim 1, wherein the composite isolation sheet comprises an inner cuff part, a front part for forming the front portion, and a back part for forming the back portion, and wherein the front part and the back part are not connected.

14. An absorbent article comprising the absorbent body of claim 1 and an application means, wherein the application means is selected from the group of a fastening means and an elastic belt.
